(19) United States
(12) Patent Application Publication
(10) Pub. No.: US 2025/0239344 A1
(43) Pub. Date: Jul. 24, 2025

(54) MEDICATION USAGE DURATION DETERMINATION

(71) Applicant: International Business Machines Corporation, Armonk, NY (US)

(72) Inventors: Uri Kartoun, Cambridge, MA (US); Jianying Hu, Bronx, NY (US); VIBHA Anand, Chestnut Hill, MA (US)

(21) Appl. No.: 18/418,953

(22) Filed: Jan. 22, 2024

(51) Int. Cl. G16H 20/10; G16H 50/30

(52) U.S. Cl. CPC G16H 20/10; G16H 50/30

(57) ABSTRACT

A processor determines use duration for a medication of interest for providing patient treatment. Using a specified duration threshold, the processor performs a data linkage assessment on a data frame that includes at least covariates and medical outcome to determine data linkage between the covariates and the medical outcome, and the specified duration threshold. Based on the data linkage, the processor determines a linkage measure value that determines strength of an effect of taking the medication of interest, on the medical outcome. Until one or more stopping criteria is met, the processor performs an iterative processing of: performing of the data linkage assessment using different specified duration threshold and filtered data frame, and determining of the linkage measure value. Responsive to the one or more stopping criteria being met, the processor identifies the current iteration's specified duration threshold as a target use duration of the medication of interest.

### BACKGROUND

The present disclosure relates generally to computers and computer applications, medical treatments, and more particularly to determining medication use duration.

### BRIEF SUMMARY

The summary of the disclosure is given to aid understanding of a computer system and method of determining medication usage duration, and not with an intent to limit the disclosure or the invention. It should be understood that various aspects and features of the disclosure may advantageously be used separately in some instances, or in combination with other aspects and features of the disclosure in other instances. Accordingly, variations and modifications may be made to the computer system and/or their method of operation to achieve different effects.

A computer-implemented method in some embodiments includes retrieving from a database, a data frame that includes at least covariates and medical outcome associated with subjects with usage history of a medication of interest. The computer-implemented method also includes performing, using a specified duration threshold, a data linkage assessment on the data frame to determine data linkage between the covariates and the medical outcome and the specified duration threshold. The computer-implemented method also includes, based on the data linkage, determining a linkage measure value that determines strength of an effect of taking the medication of interest, on the medical outcome. The computer-implemented method also includes comparing the linkage measure value with a predefined risk assessment threshold. The computer-implemented method also includes, responsive to determining that a difference between the linkage measure value and the predefined risk assessment threshold is greater than a predefined difference threshold: filtering the data frame by excluding from the data frame, data associated with subjects with use history of the medication of interest for longer than the specified duration threshold; specifying a different duration threshold as the specified duration threshold; and repeating the performing of the data linkage assessment, the determining of the linkage measure value, and the comparing of the linkage measure value with a predefined risk assessment threshold. The computer-implemented method also includes, responsive to determining that the difference between the linkage measure value and the predefined risk assessment threshold is not greater than the predefined difference threshold, identifying the specified duration threshold as a target use duration of the medication of interest. The computer-implemented method also includes providing a treatment using the medication of interest for the target use duration in treating a patient.

A computer system and a computer program product configured to achieve or cause the method described above are also disclosed herein.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 shows an example of a computing environment, which can implement medication usage duration determination in some embodiments.

FIG. 2 is a flow diagram illustrating a method of determining a threshold duration for a medication in some embodiments.

FIG. 3 is a diagram illustrating an iterative method of applying a data linkage assessment to determine prescription duration for a medication in some embodiments.

FIG. 4 is a flow diagram illustrating a method in some embodiments of determining usage or treatment duration threshold of a medication.

FIG. 5 is a diagram illustrating example results of an iterative processing for determining usage or treatment duration threshold of a medication in some embodiments.

FIG. 6 is a diagram illustrating a cohort construction in some embodiments.

FIG. 7 is a diagram showing components of a system in some embodiments that determines a duration threshold for a medication of interest.

### DETAILED DESCRIPTION

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as medication usage duration determination algorithm code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

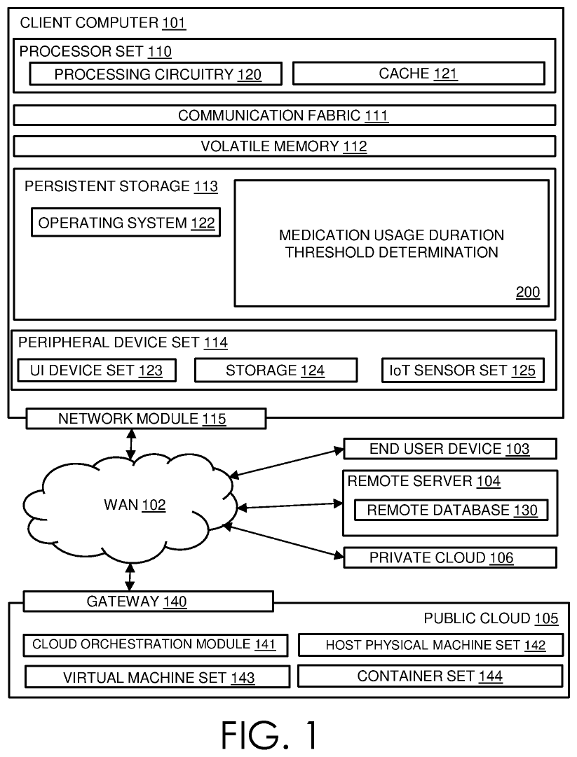

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

CLOUD COMPUTING SERVICES AND/OR MICROSERVICES (not separately shown in FIG. 1): private and public clouds 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to as “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

Medications may be harmful if taken for extended periods. For example, certain medications if taken for long durations may lead to gastrointestinal issues like ulcers and bleeding, as well as increased risk of heart problems, increase risks of dependence, and/or risks in conditions such as but not limited to osteoporosis, cataracts, elevated blood sugar, and/or others.

A data-driven computational method in some embodiments identifies a threshold for a duration of use of a medication, for example, a duration after or outside of which a prescription is no longer efficient and/or become harmful to the health of the patient. In some embodiments, the method calculates a break-even threshold, in which a use of a medication above that threshold may indicate potential harm to the patient's health.

The method may help or support a process of making a clinical decision, for example, guide clinicians on prescription length of a medication given a specific patient and the patient's conditions. The method can further guide clinicians to prescribe a medication differently for different groups of patients (e.g., different demographic groups). The method can further help in understanding the results of past clinical trials. The method can also help in understanding the safety aspects of prescribing a medication given a particular duration. In some embodiments, the method uses one or more causality inference techniques as one of the components, which makes its results and conclusions highly accurate. In some embodiments, using a causality inference technique can produce more accurate conclusion than using association-based methods.

An example use case of the method can include aiding in deciding for how long to apply an insulin therapy to a patient, as well as the type (e.g., short or long acting) of insulin. The method can be applicative to any medication therapy and type.

The method can focus on a specific medication to iteratively assess that medication's effect considering a given outcome using causality inference to determine an optimal duration threshold. For example, the method iteratively processes an observational database to determine a duration threshold for a given medication of an interest. The duration threshold indicates whether using the medication could be harmful if used longer or shorter compared to the threshold. For instance, the method may process an observational electronic health record database to identify a duration threshold, for example, identify an optimal duration threshold per a medication of an interest. Durations and duration thresholds may also be short (e.g., a few seconds, minutes, hours) as the method is also applicative to experiments conducted in a laboratory considering any type of time series, not just to data originated from observational databases.

An odds ratio (OR) is a measure of association between a treatment (e.g., taking a medication) and an outcome. The OR represents the odds that an outcome will occur given a particular treatment, compared to the odds of the outcome occurring in the absence of that treatment. For example, if the OR is 1.5, the odds of disease after being treated are 1.5 times greater than the odds of disease if one did not receive the treatment. As another example, an OR of 0.8 means there is a 20% decrease in the odds of an outcome with a given treatment compared to absence of the treatment.

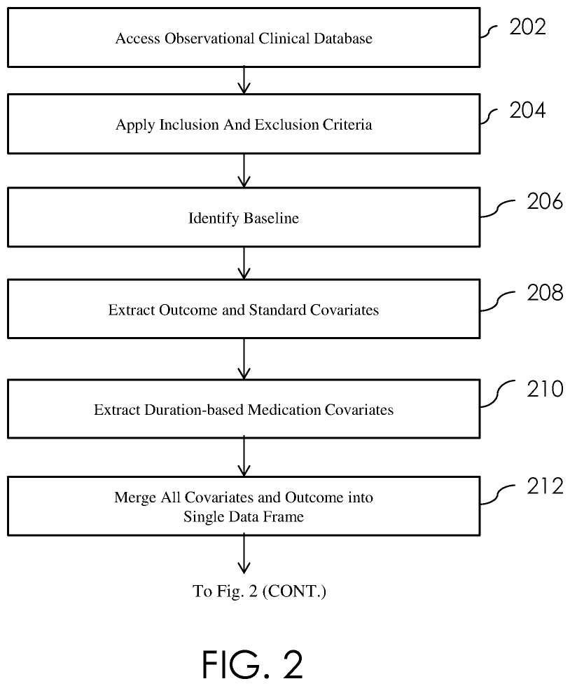

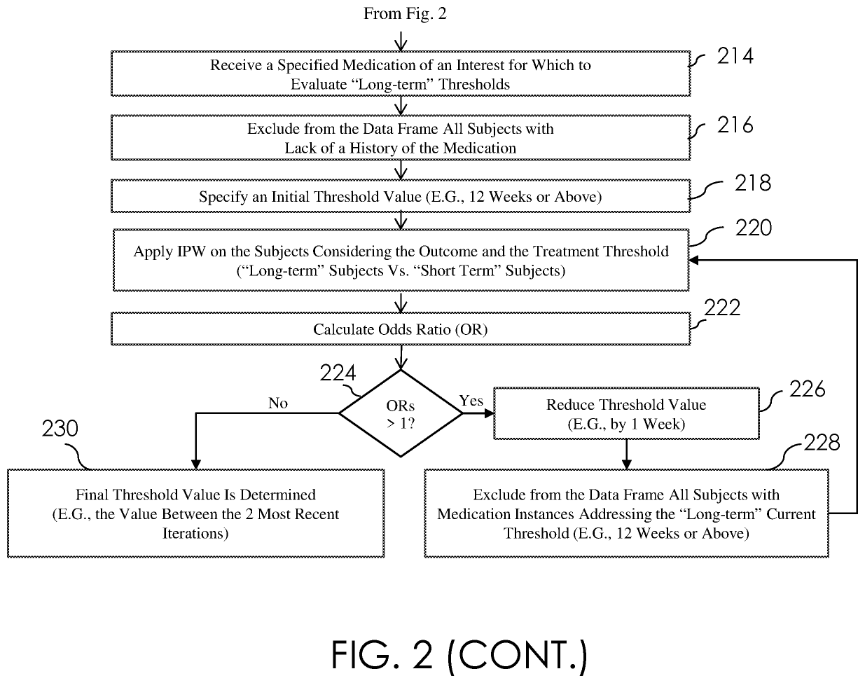

FIG. 2 is a flow diagram illustrating a method of determining a threshold duration for a medication in some embodiments. In some embodiments, the method is an iterative process that determines a duration threshold for a given medication, for example, in which consuming the medication for a longer time than the duration threshold may result in an undesirable medical outcome or health-related outcome. Undesirable medical outcome of may be a readmission, a recurrent surgery, uncontrolled medical condition (e.g., blood pressure, blood sugar level), and/or other conditions. The method can be performed by one or more hardware processors, for example, including computer processors, for example, described above with reference to FIG. 1. In the following description, the term “a processor” can mean “one or more processors,” and does not necessarily refer to a single or the same processor. For example, different processors working in a cooperative manner can perform the different steps or features of the method described in FIG. 2. Data used in the method is accessed with the permission of the owners and/or patients to whom the data belongs.

At 202, a processor accesses an observational clinical database. The data retrieved and used from the clinical database may include anonymized historical health data from hospitals and/or clinics about medication intakes and conditions of subjects taking the medication.

At 204, the processor applies inclusion and exclusion criteria to data retrieved from the observational clinical database. Inclusion and exclusion criteria can function as a filter for selecting data for use. Examples of inclusion and exclusion criteria include, but are not limited to data associated with patients experiencing conditions of interest, data associated with duration or periods of interest, and/or others. By way of example, data can be filtered by excluding patient data without a surgical procedure of interest. Then the data can be filtered further by a time restriction, for example, the data can be filtered by excluding patient data without the surgical procedure of interest during a time period (e.g., a predefined time period or time duration of interest). Another exclusion criterion can be that patient data with less than 2 years (or another time length) of interaction with a care system prior to a medical event such as a surgery is to be excluded. Yet another exclusion criterion can be to exclude patient data with no follow-up period with the care system after a medical event.

At 206, the processor identifies a baseline (referred also as index date). An example of a baseline is the first surgery in the data. In one embodiment, to identify a baseline given electronic health record database, patients with at least one surgery are identified. The identification of such patients is based using procedure codes, such as International Classification of Diseases (ICD) codes (e.g., ICD-10) or Classification of Interventions and Procedures (OPCS) codes (e.g., OCPS-4). The identification may also be based on applying a text processing algorithms on clinical narrative notes, instead of or in combination with procedure codes. The earliest code/note is then considered as candidate for baseline. As subsequent steps, patients with unqualified baselines are filtered out—for example, patients that do not meet one or more criteria at the first surgery are filtered out, as well as patients with insufficient prior data, patients with no follow-up data, etc.

At 208, the processor extracts outcome and standard covariates. Outcome informs the result of taking the medication, what conditions patients experienced after taking the medication for their prescription duration. In some embodiments, covariates that potentially affect clinical decisions are used. Examples of covariates include, but are not limited to, age, history of disease such as history of Crohn's disease (CD), history of being current or former smoker, most recent values of laboratory observations (e.g., albumin, hemoglobin), comorbidities based on Clinical Classifications Software (CCS) codes or similar disease classification methods, prior surgeries, allergies, family history of diseases, vaccinations, total number of digestive related comorbidities, total number of gastrointestinal cancers, length of Ulcerative colitis (UC) and/or CD (e.g., in months) or of any other disease. Additional covariates may be polygenic risk scores, medications, covariates indicating duration use of medication.

At 210, the processor extracts duration-based medication covariates. In some embodiments, duration-based covariates are data entries indicating prescriptions associated with durations (e.g., 30-day supply, 6-month supply, etc.).

At 212, the processor merges all covariates and outcome into a single data frame. In one embodiment, the data frame is a 2-dimensional data structure with columns and rows. The columns represent covariates (multiple) and an outcome column. Each row represents one patient. For example, the following data can be included in the columns. There can be a covariate column indicating a characteristic of a patient such as “is_X”, where X represents a characteristic, where the value for the column would include a binary value of 0 or 1, depending on whether the patient possesses that characteristic. The covariate column “is_uc”, for example, contains either 1 (patient has history of UC) or 0 (patient does not have history of UC). The laboratory covariate column albumin, for example, contains continuous values. Observation covariates, such as laboratories may be limited by time (e.g., most recent during the preceding 6 months on or before baseline). Disease covariates may be binary (patient has or does not have the condition in the patient's history) or continuous (total number of indications as documented in the patient's history information). Medication covariates may be binary (patient has or does not have an indication for the medication in the patient's history), or continuous (total number of prescriptions in the patient's entire history or in a pre-defined time period, e.g., past 2 years). The column that represents the outcome is binary-indicating if the patient has or does not have the outcome in a subsequent pre-defined period of time (follow-up), for example, within 5 years after baseline. The outcome indication may relay procedure codes or notes as described above. An additional column is the time to the outcome (e.g., considering 5 year it would be a number in the range of 1 to 1,825 days). In case a patient does not have the outcome, that number would represent loss to follow-up, i.e., calculated based on the latest date indicating an interaction with the care system (e.g., an encounter, a lab value, a medication, a procedure). As an example, a patient who does not have the outcome during the subsequent 5 years but has an indication for interacting with the care system (e.g., an encounter on record found 6 years after baseline) would have the value 1,825 in that column. As another example, a patient who does not have the outcome during the subsequent 5 years but has a last indication for interacting with the care system 2 years after the baseline but no further indications on record would have the value 730 in that column (2*365).

At 214, a medication of an interest for which to evaluate “long-term” thresholds can be specified and the processor receives this specified medication of an interest.

At 216, the processor excludes from the data frame all subjects (e.g., patient data in rows) with lack of a history of the medication.

At 218, the processor specifies an initial threshold value (e.g., 12 weeks). In some embodiments, the initial threshold value can be predefined. In some embodiments, the predefined threshold value can be determined based on prior experience or historical prescription duration.

At 220, the processor applies a data linkage assessment method on the subjects considering the outcome and the treatment threshold (“long-term” subjects vs. “short term” subjects). An example of a data linkage assessment method is causal inference-based technique such as inverse probability weighting (IPW). IPW is a statistical technique employed to address selection bias in observational studies when estimating the causal effect of an exposure, such as a medication treatment, on an outcome. The method involves weighting patients by the inverse of the probability that they would receive the treatment they received, given their covariates. This probability is known as the propensity score. By doing so, IPW creates a synthetic sample in which the treatment assignment is independent of the measured confounders, mimicking a randomized experimental design. Consider, for example, a study aiming to assess the impact of a medication on the risk of developing a specific health condition. The probability that a patient receives this new medication could be influenced by a range of factors, including patient characteristics, or prior health status. IPW would first involve modeling the probability of each patient receiving the medication based on these factors, and then assigning a weight to each patient that is the inverse of this probability. Patients who were less likely to receive the medication but did receive it are given more weight in the analysis, and vice versa. The outcome, such as the incidence of the health condition, is then analyzed using these weights to estimate what the effect of the medication would have been if the treatment had been randomly assigned. The inputs include data on patient exposure to a treatment or intervention (like a medication), the outcome of interest, and a set of covariates that are thought to influence both the exposure and the outcome (e.g., laboratory observations indicating severity in UC/CD patients, specific diagnoses such as respiratory or circulatory). For each patient in the observational study, the input includes their treatment status (whether they received the medication), the outcome data (whether the outcome occurred), and the values of confounding variables (e.g., patient characteristics, UC/CD diagnosis). A propensity score model, for example, a logistic regression, is then fitted to these inputs to calculate the probability of receiving the treatment for each patient, given the confounders. The output of IPW is a set of weights for each patient, which are used to create a weighted dataset that balances the confounders across treatment groups. Analysis of this weighted dataset yields estimates of the average treatment effect of the exposure on the outcome, which are less biased by confounding variables than unweighted estimates. Thus, the final output is a more accurate assessment of the causal effect of the medication on the health outcome. Another example of a data linkage assessment method is an association-based method such as Cox regression. Briefly, Cox regression builds a predictive model for time-to-event data. The predictive model produces a survival function that predicts the probability that the event (the outcome) of interest has occurred at a given time t for given values of the predictor variables (the covariates).

At 222, the processor calculates a linkage measure value to measure strength (e.g., how strong the effect of taking a long-term vs. short-term medication is). In some embodiments, the linkage measure value can be an OR. Within the context of medication exposure, an OR is a measure of whether taking a medicine increases or decreases the chance of the outcome. ORs after applying IPW are calculated to determine the association between receiving a treatment and an outcome, while accounting for confounding variables. After IPW is applied, each subject is assigned a weight based on the inverse probability of receiving the treatment they received, given their covariates. This creates a weighted sample that approximates a randomized experimental design. Within this weighted sample, the odds of the outcome for those who received the treatment are compared to the odds of the outcome for those who did not receive it. The odds for each group are calculated as the weighted sum of the group who had the outcome divided by the weighted sum of the group that did not have the outcome. The OR is then the ratio of these two odds. Values greater than 1 suggest a positive association between the treatment and the outcome. Values less than 1 suggest a negative association. Values equal to 1 suggest no association.

At 224, the processor compares the linkage measure value with a predefined risk assessment threshold. In some embodiments, a risk assessment threshold is odds ratio threshold. For instance, in some embodiments, the predefined risk assessment threshold can be expressed as an odds ratio value of 1 (e.g., odds ratio=1). For example, if the linkage measure value represented by odds ratio is greater than 1, an iterative processing is performed at 226, otherwise, the method proceeds to 230. In some embodiments, risk assessment threshold is difference in incidence.

In some embodiments, at 224, the processor evaluates the value difference between the linkage measure value and the assessment threshold value. If no significant difference is found (e.g., even if the linkage measure value is greater than the assessment threshold value), the method proceeds to 230, and the processor declares the current treatment duration threshold value as the final value,

At 224, if a significant difference is found (or e.g., the linkage measure value represented by odds ratio is greater than 1), then the method proceeds to 226. At 226, the processor specifies a threshold duration with a different value (e.g., one week less to modify 12 to 11 weeks, or another reduced duration value). For example, in some embodiments, the different value is a threshold with a lower value than the threshold being assessed in the current iteration (e.g., 1 day lower, 1 week lower). In some embodiments, specifying a threshold value with a different may include specifying a threshold with a higher value than the threshold being assessed in the current iteration (e.g., 1 day higher, 1 week higher).

At 228, the processor excludes from the data frame all subjects with usage duration of the medication that is above the current reduced threshold value. The processor then iterates the processing by proceeding to 220 and repeating the processing of 220, 222, and 224. At 224, the odds ratio value calculated at 222 is compared to a threshold (e.g., 1). If the current odds ratio value is equal or below the odds ratio threshold the process proceeds to 230 (i.e., final duration threshold value was determined). If the current odds ratio value is above the odds ratio threshold the process proceeds to 226 and 228 towards an additional iteration (220, 222, and 224).

In some embodiments, the single data frame at 212 can be split to include subpopulations (e.g., subjects of specific characteristic, subjects with history of inflammatory conditions, subjects with no inflammatory conditions, age range, and/or another subgroup). In some embodiments, the iterative processing of 214-230 is applied on each subpopulation separately. In this way, the processor assesses whether the medication threshold varies across subpopulations; the medication may be safe to use for a very long time for subjects for a first subpopulation and safe to use for a short time for a second subpopulation.

In some embodiments, the step of stopping and declaring current treatment threshold value as the final value at 230 may be optional. In some embodiments, a different stopping criterion may apply, such as, but not limited to: stopping after a pre-defined number of iterations such as 1,000, stopping when the linkage measurement value (e.g., odds ratios) converge to a pre-defined value (e.g., close to 0, very high value such as 10), stopping when it is not possible to apply IPW given a small number of subjects, since the number of subjects decreases in each iteration.

The method in some embodiments identifies for a given medication a duration threshold in which: it could be harmful to take beyond the threshold, and/or it could be inefficient to take it further. In some embodiments, pharmaceutical organizations may use the method to gather insight for past clinical trial designs (e.g., explore trial end points, explore subgroups where different thresholds apply) and/or to establish medications. Healthcare institutions such as hospitals, health governing agencies and/or health insurance companies also may use the method to determine appropriate medication durations for a given medication.

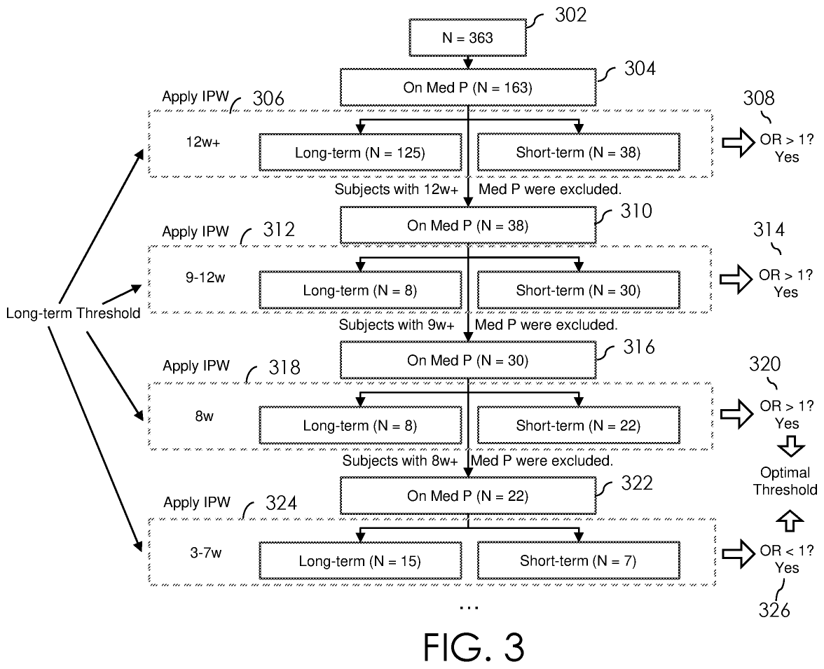

FIG. 3 is a diagram illustrating an iterative method of applying a data linkage assessment to determine prescription duration for a medication in some embodiments. The method can be performed by or on one or more computer processors, for example, shown in but not limited to, FIG. 1. At 302, data that has been filtered is used. For example, it may be that there are N=363 subjects who meet the inclusion criteria and exclusion criteria, for example, as described above with reference to FIG. 2. Data that is associated with those subjects are used initially. At 304, the data is further filtered to include only those of the subjects who are currently on a medication that is specified as medication of interest. By way of example, consider that 163 out of 363 are currently on the specified medication, and therefore, N becomes 163 (N=163). An initial threshold value that is specified is used to apply a causality inference technique. For example, out of 163, those subjects that were on the specified medication for the initial threshold value is used. Consider that in this example, the initial threshold value is 12+ weeks (12 weeks or more). Consider also in this example, that out of 163 subjects, 125 subjects (N=125) were on the specified medication for 12+ weeks, and that 38 subjects (N=38) were on the specified medication for less than 12 weeks.

At 306, a data linkage assessment method such as IPW is performed iteratively. In a first iteration, an initial long-term duration threshold value is defined (e.g., 12+ weeks). Patients on Med P (any duration prescription lengths) are considered while patients with no Med P prescriptions in their history are excluded (excluding 200 such patients, by way of example, results 163 patients, as shown in 304). A processor applies IPW on the subjects (N=163), including the long-term and the short-term patients (N=125 and N=38, respectively). Note that the long-term group (N=125) received at least one prescription of Med P of a duration of 12 weeks or more, while the short-term group (N=38) received at least one prescription of Med P but with durations that are shorter than 12 weeks. After applying IPW on the 163-patient population, odds ratio value is calculated and compared to an odds ratio threshold value (e.g., 1). If the calculated odds ratio value is above the odds ratio threshold value (e.g., equals to 3) then a second iteration is applied. In the second iteration all patients who took at least one prescription of Med P that exceeded the current duration threshold (12+ weeks) were excluded (N=125). As such only the patients who were considered short-term in the first iteration (taking Med P for less than 12 weeks) are incorporated into the second iteration (N=38). In the second iteration also a revised long-term duration threshold value is defined (e.g., 9 to 12 weeks), replacing the long-term duration threshold used in the first iteration (12+ weeks). This group (N=38) is then split to long-term and short-term groups based on the revised duration threshold. Of the 38 patients, 8 are considered long-term (at least one Med P associated with a duration of 9 to 12 weeks) while the rest 30 are considered short-term (received at least one prescription of Med P but with durations that are shorter than 9 weeks). After applying IPW on the 38-patient population, odds ratio value is calculated and compared to an odds ratio threshold value (e.g., 1). If the odds ratio value is above the odds ratio threshold value (e.g., equals to 2), then a third iteration is applied in a similar manner. The process proceeds with additional iterations in which in each iteration patients associated with durations of Med P that exceed a revised long-term duration threshold are filtered out, applying IPW on the smaller population, calculating an odds ratio value, and comparing the odds ratio value to an odds ratio threshold value (e.g., 1). The process ends when a stopping criterion is addressed (e.g., odds ratio=0.9, below the odds ratio threshold value).

To highlight the process further, the processor compares the linkage measure value with a predefined risk assessment threshold. For instance, the predefined risk assessment threshold can be an odds ratio value of 1. At 308, the processor determines whether the calculated odds ratio is greater than the odds ratio value of 1. If the calculated odds ratio is greater than the odds ratio value of 1, the data frame or data set is filtered by excluding data of the subjects with instances of use of the medication above the threshold of 12 weeks. In this example, 125 subjects are excluded, leaving N=38 subjects with less than 12+ weeks. Using this data frame 310, the process of applying a data linkage assessment method and calculating a linkage measure value repeats at 312. A different threshold value is specified, for example, 9 weeks. Consider that in this example (as shown at 312) there are N=8 subjects with 9 to 12 weeks of use of the medication (since the data excluded 12+ weeks of usage data), N=8 subjects are those that have 9-12 weeks of the medication usage), and N=30 subjects with less than 9 weeks of the medication usage.

In some embodiments, the processor evaluates the value difference between the linkage measure value and the predefined risk assessment threshold, and if there is no significant difference (e.g., determined by another threshold), the iterative process stops, and determines that the current treatment duration threshold to be the final duration threshold. If a significant difference is determined, then additional iterations are processed. For example, at 314, the processor determines whether the calculated odds ratio is greater than the odds ratio value of 1. If the calculated odds ratio is greater than the odds ratio value of 1, the data frame or data set is filtered by excluding data of the subjects with instances of use of the medication above the threshold of 9 weeks. In this example, 8 subjects are excluded, leaving N=30 subjects with less than 9+ weeks. Using this data frame 316, the process of applying a data linkage assessment method and calculating a linkage measure value repeats at 318. A different threshold value is specified, for example, 8 weeks. Consider that in this example (as shown at 318) there are N=8 subjects with 8+ (8 or more) weeks of use of the medication, and N=22 subjects with less than 8 weeks of the medication usage.

At 320, the processor determines whether the calculated odds ratio is greater than the odds ratio value of 1. If the calculated odds ratio is greater than the odds ratio value of 1, the data frame or data set is filtered by excluding data of the subjects with instances of use of the medication on or above the threshold of 8 weeks. In this example, 8 subjects are excluded, leaving N=22 subjects less than 8 weeks of the medication usage. Using this data frame 322, the process of applying a data linkage assessment method and calculating a linkage measure value repeats at 324. A different threshold value is specified, for example, 3 to 7 weeks. Consider that in this example (as shown at 324) there are N=15 subjects with 3 to 7 weeks of use of the medication, and N=7 subjects with less than 3 weeks of the medication usage.

At 326, the processor again compares the linkage measure value with the predefined risk assessment threshold, e.g., the processor determines whether the calculated odds ratio is greater than the odds ratio value of 1. Consider that in this example, at this iterative stage, the odds ratio is less than 1, that is, the linkage measure value is less than the predefined risk assessment threshold. As a result, the processor stops the iterative process. In some embodiment, the processor may determine that the duration between the current usage duration threshold (e.g., 3 to 7 weeks) and the prior iteration's usage duration threshold (e.g., 8 weeks) to be an optimal duration for usage of the medication.

Once an optimal threshold for duration of the medication's usage is determined, the method can also include treating a patient based on the determined duration of the medication's usage, for example, treating a patient with the medication for that amount of time, or for a shorter duration. Treating a patient includes giving the patient the medication, or causing the medication to be given to the patient, for the determined duration or for a shorter than the determined duration.

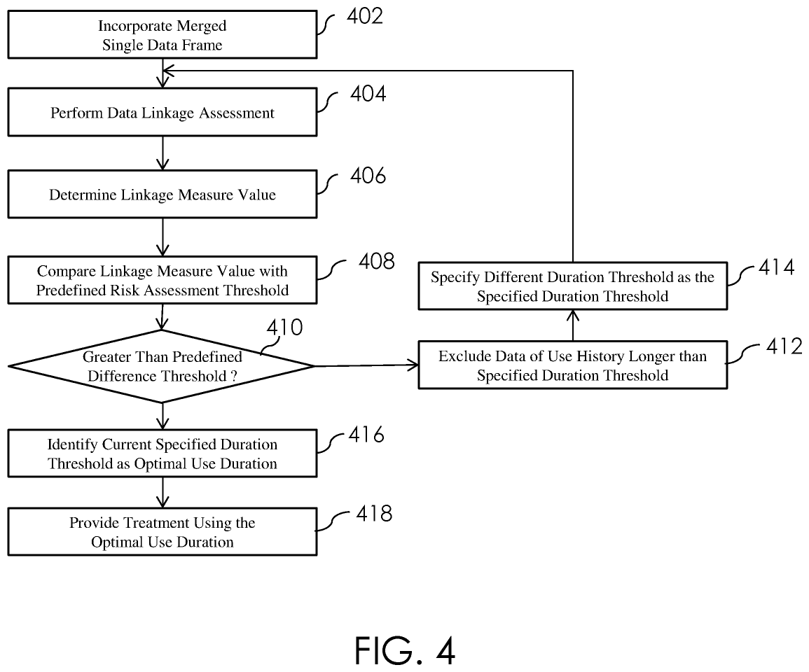

FIG. 4 is a flow diagram illustrating a method in some embodiments of determining usage or treatment duration threshold of a medication, the duration threshold over which consuming the medication may result in an undesirable medical outcome. Examples of undesirable medical outcome may be a readmission, a recurrent surgery, uncontrolled medical condition (blood pressure, blood sugar level), and/or others. The method can be performed by or run on one or more hardware processors such as computer processors, for example, but not limited to those described with reference to FIG. 1 above. At 402, data related to patient history is incorporated (e.g., as shown at 212). At 402, a data frame that includes covariates and medical outcome is retrieved or received from a database. For example, as described with reference to 212 in FIG. 2, the data frame includes merged covariates and outcome. For example, the data frame includes covariates of an observational clinical database about patients with history of usage of a medication of interest (e.g., specified medication). The medication of interest or specified medication can be specified by a subject matter expert, an administrator, a user, and/or based on historical or experimental values. In some embodiments, the covariates include at least duration-based covariate data indicating prescriptions associated with durations (e.g., 30-day supply, 6-month supply, etc.). In some embodiments, the covariates include indications for disease severity (e.g., laboratory-based, comorbidity-based, patient characteristic, polygenic risk scores), diagnoses, observations, procedures, risk scores, and demographics. In some embodiments, the data frame split into subpopulations before being used in the subsequent processing shown in FIG. 4. For example, the data frame is split by different characteristics such as but not limited to, history of inflammatory conditions vs. no history of inflammatory conditions, and/or others. In some embodiments, subsequent iterative processing shown in FIG. 4 can be applied on each subpopulation separately to assess whether the optimal duration medication threshold varies across subpopulations; for example, whether the medication is safe to use in long-term for subjects for a first subpopulation and safe to use for shorter time for a second subpopulation.

At 404, using a specified duration threshold, a data linkage assessment is performed on the data frame to determine data linkage between a medical outcome and the specified duration threshold value. For example, initially, a duration threshold can be specified for starting an iterative process, for example, as described above with reference to FIG. 2, and the data linkage assessment performed based on this initial duration threshold. An example of the data linkage assessment includes a regression algorithm such as a Cox regression. Another example of the data linkage assessment includes causal inference technique such as IPW.

At 406, based on the data linkage, a linkage measure value that determines a strength of an effect of taking the medication of interest, on the medical outcome, is determined. An example of the linkage measure is odds ratio. For example, the linkage measure value includes an odds ratio value. For this example, the risk assessment threshold is also expressed in terms of odds ratio. In another embodiment, the risk assessment threshold is expressed as a difference in incidence.

At 408, the linkage measure value that is calculated is compared with a predefined risk assessment threshold. Responsive to determining that a difference between the linkage measure value and the predefined risk assessment threshold is greater than a predefined difference threshold, the following processing can be performed: filtering the data frame by excluding from the data frame, data associated with subjects with use history of the medication of interest for longer than the specified duration threshold; specifying a different duration threshold as the specified duration threshold; and repeating the performing of the data linkage assessment, the determining of the linkage measure value, and the comparing of the linkage measure value with a predefined risk assessment threshold. For instance, at 410, if a difference between the linkage measure value and the predefined risk assessment threshold is greater than a predefined difference threshold, the processing proceeds to 412. The predefined difference threshold can be configured or defined. Such difference threshold indicates an insignificant difference between the linkage measure value and the predefined risk assessment threshold. At 412, the data frame is filtered by excluding from the data frame, data associated with subjects with use history of the medication of interest for longer than the specified duration threshold. At 414, a different duration threshold is specified as the specified duration threshold. In some embodiments, the different duration threshold that is specified is a threshold with a lower value than the threshold being assessed in the current iteration (e.g., 1 day lower, 1 week lower). In other embodiments, the different duration threshold that is specified may be a threshold with a higher value than the threshold being assessed in the current iteration (e.g., 1 day higher, 1 week higher). The process than iterates back to 404, where a subset of the data frame and different duration threshold are used in repeating the processing at 404.

At 410, responsive to determining that the difference between the linkage measure value and the predefined risk assessment threshold is not greater than the predefined difference threshold, the processing proceed to 416. At 416, the specified duration threshold (of the current iteration) is identified as a use duration of the medication of interest, e.g., as a target use duration of the medication of interest. For instance, at 410, if it is determined that the difference between the linkage measure value and the predefined risk assessment threshold is not greater than the predefined difference threshold, the iterative processing of 404 to 414 stops. The specified duration threshold used in the current iteration can be considered an optimal use duration, and issued as a target duration of use for the medication of interest.

At 418, a treatment is provided to a patient using the medication of interest for the target use duration, identified at 416, in treating the patient. For instance, a patient is given the medication of interest for the target use duration during a medical treatment. Since the method determined that using the medication beyond the identified optimal duration of use could be ineffective or not beneficial to the health of the patient, the patient is not treated with the medication of interest beyond that target use duration.

In some embodiments, at 410, different or additional stopping criterion may stop the iterative processing of 404 to 414. Examples of such additional stopping criterion includes, but are not limited to, stopping after a pre-defined number of iterations have been performed such as 1,000, stopping when the odds ratios converge to a pre-defined convergence value (e.g., close to 0, or a relatively high value such as 10), stopping based on the data size of the covariates, e.g., when it is not possible to apply IPW given small number of data remaining (e.g., since the size of the data frame decreases in each iteration).

Values or such as thresholds, e.g., predefined risk assessment threshold, predefined difference threshold, specified duration threshold, different duration threshold, described above can be configured by a subject matter expert, an administrator, a user, and/or based on historical or experimental values.

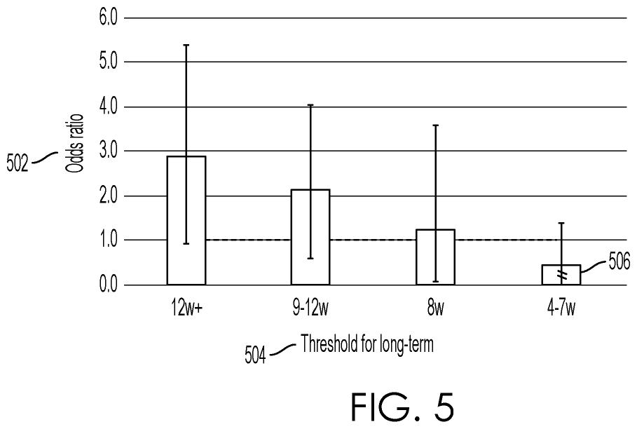

FIG. 5 is a diagram illustrating example results of an iterative processing described above in some embodiments. For instance, consider that the linkage measure value used in the processing is in terms of odds ratio. The y-axis 502 of the diagram shows odds ratio values, e.g., 0.0 to 6.0, and the x-axis of the diagram 504 shows the duration threshold values, e.g., 12+ weeks, 9-12 weeks, 8 weeks and 4-7 weeks. In this example, a stopping criterion is that the linkage measure value, expressed in terms of odds ratio is less than 1. Odds ratio value is less than one in this example for the duration threshold value that is 4-7 weeks, as shown at 506. Therefore, in this example scenario, the use duration (e.g., considered as an optimal duration) is estimated to be in the range of 4 to 8 weeks. Optionally confidence intervals (e.g., 95%) are calculated for each odds ratio value as shown in FIG. 5 as vertical lines.

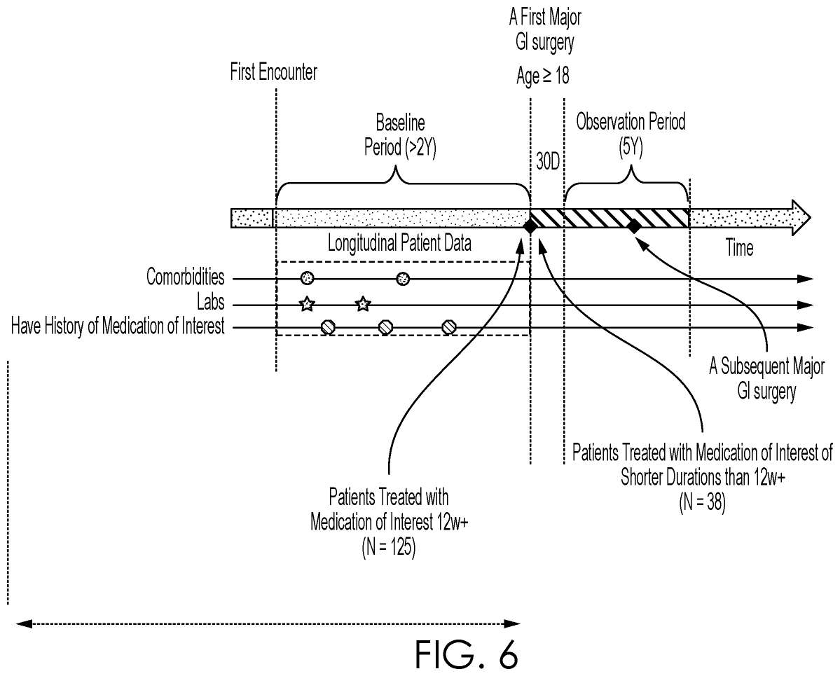

FIG. 6 is a diagram illustrating a cohort construction in some embodiments. The figure indicates an example in which the date of a first major gastrointestinal (GI) surgery is identified and defined as baseline. All such patients must be adults, however, in additional embodiments, any other age restrictions may be applied or entirely unrestricted. All such patients are guaranteed to have at least 2 years of prior longitudinal data to baseline. This is achieved by identifying for each patient a data entry indicating an encounter with the care system, occurring at least 2 years before baseline (e.g., an outpatient visit, a procedure, a vital sign, etc.). The 2 years definition is configurable and may be of any length. Covariates then are extracted on or prior to baseline, as described in the above paragraphs. Covariates indicating prescription of the medication of interest are extracted as well, for example, binary, continuous. Such covariates may include an indication of whether the patient has ever been prescribed the medication of interest, total number of prescriptions of the medication of interest, total number of prescriptions of the medication of interest associated also with length of use (e.g., 1 day, 3-7 weeks, 12+ weeks, etc.). A certain medication of interest may be associated with multiple prescription covariates, each indicating a use or uses for different duration lengths. An outcome of an interest is assessed and extracted within a pre-defined follow-up period occurring after baseline (e.g., up to 5 years). The follow-up period length is configurable. The outcome may be a clinical event as described in previous sections. Aspects of loss to follow-up are addressed as well, as described in previous sections. Outcome events may be considered from a certain time after baseline, not immediately, for example, after 30 days and up to 1,825 days (5 years). This is to account for identification of a true outcome (e.g., a second major surgery), as opposed to an event that is considered as part of the baseline event (e.g., an admission that lasted up to a few days after the baseline). Once all the above has been addressed, a single data frame is formed and is used for further analyses (e.g., as described with reference to FIG. 2 at 212). The groups highlighted in FIG. 6 (N=125 and N=38) indicate two groups of patients. The first group (N=125) is associated with long-term use of the medication of interest (12 weeks or more) while the second group (N=38) is associated with short-term use of the medication of interest (less than 12 weeks). All 163 patients had at least one prescription of the medication of interest in their history, while 200 of 363 patients did not have any past indication for the medication of interest. This refined data frame of 163 patients is then used to apply additional steps, such as IPW, odds ratio calculations, comparison of calculated odds ratio to an odds ratio threshold, determination of a revised prescription duration threshold, performance of additional iterations, or stopping and declaring a prescription duration threshold as a final value.

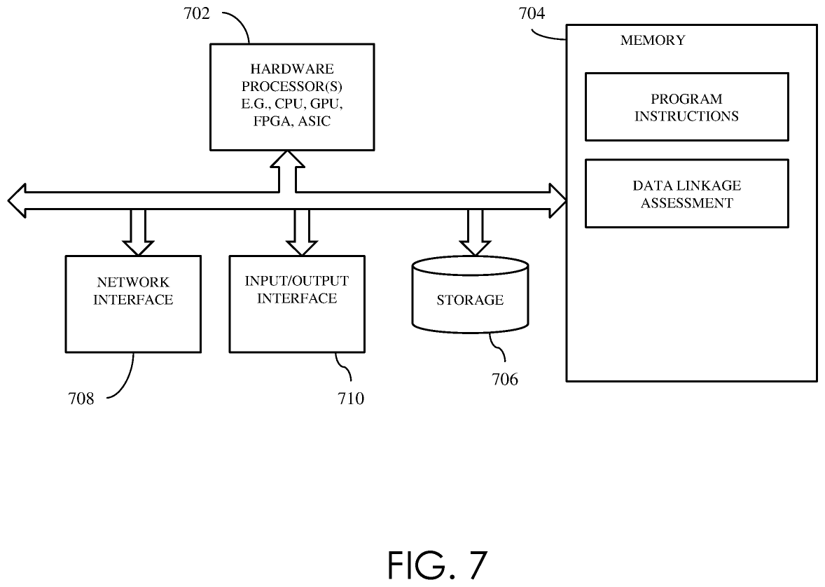

FIG. 7 is a diagram showing components of a system in some embodiments that determines a duration threshold for a medication of interest. One or more hardware processors 702 such as a central processing unit (CPU), a graphic process unit (GPU), and/or a Field Programmable Gate Array (FPGA), an application specific integrated circuit (ASIC), and/or another processor, may be coupled with a memory device 704, and determine use duration for a medication of interest (e.g., specified medication). A memory device 704 may include random access memory (RAM), read-only memory (ROM) or another memory device, and may store data and/or processor instructions for implementing various functionalities associated with the methods and/or systems described herein. One or more processors 702 may execute computer instructions stored in memory 704 or received from another computer device or medium. A memory device 704 may, for example, store instructions and/or data for functioning of one or more hardware processors 702, and may include an operating system and other program of instructions and/or data. One or more hardware processors 702 may receive input that includes covariates of observational clinical database. For instance, at least one hardware processor 702 may perform processing described with reference to FIGS. 2, 3 and/or 4, and determine a use duration for a medication of interest. Data used by one or more hardware processors 702 may be stored in a storage device 706 or received via a network interface 708 from a remote device, and may be temporarily loaded into a memory device 704 for determining use duration of a medication. One or more hardware processors 702 may be coupled with interface devices such as a network interface 708 for communicating with remote systems, for example, via a network, and an input/output interface 710 for communicating with input and/or output devices such as a keyboard, mouse, display, and/or others.

The terminology used herein is for the purpose of describing particular embodiments only and is not intended to be limiting of the invention. As used herein, the singular forms “a”, “an” and “the” are intended to include the plural forms as well, unless the context clearly indicates otherwise. As used herein, the term “or” is an inclusive operator and can mean “and/or”, unless the context explicitly or clearly indicates otherwise. It will be further understood that the terms “comprise”, “comprises”, “comprising”, “include”, “includes”, “including”, and/or “having,” when used herein, can specify the presence of stated features, integers, steps, operations, elements, and/or components, but do not preclude the presence or addition of one or more other features, integers, steps, operations, elements, components, and/or groups thereof. As used herein, the phrase “in some embodiments” does not necessarily refer to the same embodiment, although it may. As used herein, the phrase “in one embodiment” does not necessarily refer to the same embodiment, although it may. As used herein, the phrase “in another embodiment” does not necessarily refer to a different embodiment, although it may. Further, embodiments and/or components of embodiments can be freely combined with each other unless they are mutually exclusive.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method, comprising:
retrieving from a database, by a processor set, a data frame that includes at least covariates and medical outcome associated with subjects with usage history of a medication of interest;
performing by the processor set, using a specified duration threshold, a data linkage assessment on the data frame to determine data linkage between the covariates and the medical outcome, and the specified duration threshold;
based on the data linkage, determining by the processor set a linkage measure value that determines strength of an effect of taking the medication of interest, on the medical outcome;
comparing by the processor set, the linkage measure value with a predefined risk assessment threshold;
responsive to determining that a difference between the linkage measure value and the predefined risk assessment threshold is greater than a predefined difference threshold:
filtering by the processor set, the data frame by excluding from the data frame, data associated with subjects with use history of the medication of interest for longer than the specified duration threshold;
specifying by the processor set, a different duration threshold as the specified duration threshold; and
repeating by the processor set, the performing of the data linkage assessment, the determining of the linkage measure value, and the comparing of the linkage measure value with a predefined risk assessment threshold;
responsive to determining that the difference between the linkage measure value and the predefined risk assessment threshold is not greater than the predefined difference threshold, identifying by the processor set, the specified duration threshold as a target use duration of the medication of interest; and
providing a treatment using the medication of interest for the target use duration in treating a patient.

2. The method of claim 1, the covariates include at least duration-based covariate data indicating prescriptions associated with durations.

3. The method of claim 1, wherein the data linkage assessment includes a regression algorithm.

4. The method of claim 1, wherein the data linkage assessment includes a casual inference technique.

5. The method of claim 1, wherein the linkage measure value includes an odds ratio value.

6. The method of claim 1, wherein the risk assessment threshold includes a value expressed in terms of odds ratio.

7. The method of claim 1, wherein the risk assessment threshold is expressed as a difference in incidence.

8. The method of claim 1, wherein the different duration threshold that is specified is a lower value than the specified duration threshold being assessed in a current iteration.

9. The method of claim 1, wherein the different duration threshold that is specified is a threshold with a higher value than the specified duration threshold being assessed in a current iteration.

10. The method of claim 1, wherein the covariates are split into subpopulations before being used separately in performing the data linkage assessment in iterations.

11. The method of claim 1, wherein iterative processing applied to the data frame that includes the repeating of the performing of the data linkage assessment, the determining of the linkage measure value, and the comparing of the linkage measure value with a predefined risk assessment threshold, is stopped further responsive to determining that a stopping criterion is met.

12. The method of claim 11, wherein the stopping criterion includes a pre-defined number of iterations having been performed.

13. The method of claim 11, wherein the stopping criterion includes odds ratios converge to a pre-defined convergence value.

14. The method of claim 11, wherein the stopping criterion includes data size of the data frame.

15. A computer program product comprising:
a set of one or more computer readable storage media; and
program instructions, collectively stored in the set of one or more storage media, for causing a processor set to perform the following computer operations:
retrieve from a database, a data frame that includes at least covariates and medical outcome associated with subjects with usage history of a medication of interest;
perform, using a specified duration threshold, a data linkage assessment on the data frame to determine data linkage between the covariates and the medical outcome, and the specified duration threshold;
based on the data linkage, determine a linkage measure value that determines strength of an effect of taking the medication of interest, on the medical outcome;
compare the linkage measure value with a predefined risk assessment threshold;
responsive to determining that a difference between the linkage measure value and the predefined risk assessment threshold is greater than a predefined difference threshold:
filter the data frame by excluding from the data frame, data associated with subjects with use history of the medication of interest for longer than the specified duration threshold;
specify a different duration threshold as the specified duration threshold; and
repeat performing of the data linkage assessment using the specified duration threshold and the data frame that is filtered, determining of the linkage measure value, and comparing of the linkage measure value with the predefined risk assessment threshold;
responsive to determining that the difference between the linkage measure value and the predefined risk assessment threshold is not greater than the predefined difference threshold, identify the specified duration threshold as a target use duration of the medication of interest; and
provide a treatment using the medication of interest for the target use duration in treating a patient.

16. The computer program product of claim 15, the covariates include at least duration-based covariate data indicating prescriptions associated with durations.

17. The computer program product of claim 15, wherein the data linkage assessment includes a regression algorithm.

18. The computer program product of claim 15, wherein the data linkage assessment includes a causal inference technique.

19. The computer program product of claim 15, wherein the linkage measure value includes an odds ratio value.

20. A system comprising:
a processor set;
a set of one or more computer-readable storage media; and
program instructions, collectively stored in the set of one or more computer-readable storage media, for causing the processor set to perform the following computer operations:
retrieve from a database, data frame that includes at least covariates and medical outcome associated with subjects with usage history of a medication of interest;
perform, using a specified duration threshold, a data linkage assessment on the data frame to determine data linkage between the covariates and the medical outcome, and the specified duration threshold;
based on the data linkage, determine a linkage measure value that determines strength of an effect of taking the medication of interest, on the medical outcome;
compare the linkage measure value with a predefined risk assessment threshold;
responsive to determining that a difference between the linkage measure value and the predefined risk assessment threshold is greater than a predefined difference threshold:
filter the data frame by excluding from the data frame, data associated with subjects with use history of the medication of interest for longer than the specified duration threshold;
specify a different duration threshold as the specified duration threshold; and
repeat performing of the data linkage assessment using the specified duration threshold and the data frame that is filtered, determining of the linkage measure value, and comparing of the linkage measure value with the predefined risk assessment threshold;
responsive to determining that the difference between the linkage measure value and the predefined risk assessment threshold is not greater than the predefined difference threshold, identify the specified duration threshold as a target use duration of the medication of interest; and
provide a treatment using the medication of interest for the target use duration in treating a patient.
